(19) United States
(12) Patent Application Publication
(10) Pub. No.: US 2006/0030818 A1
(43) Pub. Date: Feb. 9, 2006

(54) System and method for securing a medical access device

(76) Inventor: Robert D. McVey, Arlington Heights, IL (US)

Correspondence Address: Bell, Boyd & Lloyd LLC, P.O. Box 1135, Chicago, IL, 60690-1135, US

(21) Appl. No.: 11/188,447

(22) Filed: Jul. 25, 2005

Related U.S. Application Data
(60) Provisional application No. 60/599,895, filed on Aug. 9, 2004.

(51) Int. Cl. A61M 5/32

(52) U.S. Cl. 604/175; 604/104; 606/192

(57) ABSTRACT

The present invention is related to a system and method of securing or retaining a medical access device in a desired position using a retention member or bolster having a diameter at least four time the diameter of the tube portion of the medical access device. In one embodiment, the medical access device is a percutaneous endoscopic colostomy (PEC) tube which has an internal bolster with a diameter over five times the diameter of the PEC tube. The increased diameter of the bolster distributes the tension over a greater surface area to minimize complications such as necrosis associated with long-term implantation of the device. The increased diameter also promotes adhesion of the layers traversed by the medical device to prevent leakage around the PEC stoma.

### PRIORITY CLAIM

This patent application is a non-provisional application of, and claims priority to and the benefit of, U.S. Patent Application Ser. No. 60/599,895, filed on Aug. 9, 2004, the entire contents of which are incorporated herein.

### BACKGROUND OF THE INVENTION

The present invention is related to a system and a method for securing a medical access device in an operable position to selectively deliver substances to or from a desired location in a body. In particular, the present invention provides an improved retention member for securing or retaining a medical access device against a surface of a body.

Progress in medical science and medical devices has enabled care providers to invasively control, assume or supplement the function of systems in an animal or human. Such progress is related to the advancements made in achieving relatively long-term access to internal structures of the body through which substances can be introduced or removed. For example, if a subject is dehydrated, an electrolyte solution can be artificially infused into the vein of a subject to correct the fluid imbalance. Air can be introduced into the lungs of a subject who cannot breathe independently. Food can be artificially introduced into the gastrointestinal tract of a subject who is incapable of eating.

Access to the internal structures of the body is often accomplished through a medical access device. The medical access device comes in many forms depending on the anatomy or the physiology of the site at which the medical access device performs its function. Although many medical access devices use existing orifices of the body to access the internal components of the body, some medical access devices are adapted to access the internal components of the body through the skin. In many cases, access through the skin is a more direct route to the accessed portion of the body than through a natural orifice. In addition, access through the skin does not preclude or disrupt the normal function that would otherwise occur if the medical access device entered the body at a natural orifice. Eliminating obtrusive access at a natural orifice is even more important when a medical access device must remain in place for an extended period of time.

Entry into the body through the skin presents a number of challenges. Natural orifices of the body are designed to provide physical and immunological barriers to invading organisms or substances. In contrast, artificial entry into the body through the skin compromises the natural barrier provided by the skin. Insertion of a medical access device through a passage or stoma created by an incision in the skin, for instance, exposes internal tissues to an immunologic challenge from outside the body of the subject which those tissues are not designed to handle. Such exposure can result in infection if not properly prevented or treated. Moreover, it may be necessary to penetrate other body cavities or internal structures such as tissues, organs, etc. to access the desired location with the medical access device. In addition, penetration of such structures or internal body cavities may expose internal tissues to other immunologic challenges associated with non-sterile environments within the body.

The function of many internal components of the body are defined by the structure of those components. FIG. 1 illustrates for example, the wall 42 of the colon or large intestine which includes different distinct layers 41, 43, and 45 that each contribute to the function of the colon. The mucosal layer 41 constitutes the lining of the colon and is responsible for the secretion of mucus which acts as a lubricant during the transport of intestinal contents. The structure of the cells lining the internal wall 42 of the colon also contribute to its absorption function. Goblet cells and blood vessels such as veins 38 present in the sub-mucosal layer 43 of the colon are involved in perfusing the colon with blood and immune factors necessary to confront the non-sterile environment of the lumen 44 of the colon. The colon also includes a muscularis externa layer 45 which comprises an inner circular layer of muscle and an outer longitudinal muscle layer. These muscle layers work in concert to move bowel contents through the colon. In addition, the mammalian colon is highly innervated by both the enteric and autonomic nervous systems. The enteric nerves control mucus secretion of the mucosal layer 41, blood flow and immune function of the submucosal layer 43, and motility of the muscles of the muscularis externa layer 45. The autonomic nervous system regulates the enteric nervous system to modify colonic function in response to central stimuli.

Disruption and destruction of the structure of the internal components of the body compromises the function and, ultimately, the viability of these components. For example, obstruction or partial obstruction of the colon compresses the structure of blood results, nerves and other cells in the layers of the wall 42 of the colon which disrupt the critical function of each of those layers. Compression of the mucosal layer 41 and muscle layers 45, for instance, can disrupt mucus flow and coordinated muscle contraction that could otherwise contribute to the resolution of the obstruction. Disruption of the flow of blood in the sub-mucosal layer 43 leads to ischemic injury and death or necrosis of the cells in each of the cell layers. Death of the cells, in turn, can cause the wall 42 of the colon to be weakened and to eventually perforate exposing the sterile environment of the peritoneal spaces surrounding the colon to non-sterile contents of the bowel. Widespread infection can follow leading to the death of the subject from untreatable sepsis.

Complications arising from disruption of the structure of internal components of the body can also occur when securing a medical access device in position within the internal component, especially if the device is retained for a long period of time. Generally, medical access devices include a tube to carry a substance into the site of action or deposit, such as the administration of nutritional formula into the stomach of a subject receiving enteral nutrition, or to drain the site of any bodily substances, such as a suprapubic catheter used to drain urine from the bladder. In addition, a medical access device can include an outflow port in the distal end of the tube and an administration set connector attached to the proximal end of the tube outside of the subject.

Once a medical access device is advanced into the space or location of its operation, the device must be secured or retained in place. In this regard, the medical access device includes a retention member or bolster usually associated with the distal end of a tube delivering a substance to, or draining a substance from, the accessed location.

One example of a medical access device is a percutaneous endoscopic colostomy tube or PEC tube. The PEC procedure is an endoscopic procedure that allows access to the bowel for colonic irrigation. This procedure offers a minimally invasive alternative to open resection in treating patients who suffer from colon dysfunctions such as volvulus, pseudo-obstruction and evacuation disorders and who are unfit for surgical intervention or have not responded to other treatment options. The technique was developed based on a similar technique of percutaneous endoscopic gastrostomy PEG providing access to the stomach for parenteral gastric feeding through a PEG tube. Accordingly, the role of the retention member of a medical access device, such as a PEG or PEC tube, includes securing the medical access device in an operable position to selectively deliver substances to or from a desired location and preventing leakage of substances to or from an undesired location.

A retention member can be a pre-formed structure or an inflatable balloon which can be physically deformed to permit insertion or removal. Retention members also can include external retention members which secure the medical access device at the level of the skin of the subject. Once the medical access device is inserted, the retention member is adapted (e.g. expanded, made firm) to provide a rigid anchor to secure the medical access device in place.

Existing retention members include an outside diameter which is less than four times the outside diameter of a tubular member with which the retention member is associated. FIG. 2, for example, illustrates an internal bolster 21 of a PEG tube well known in the art having an outer diameter 23 of less than four times the outer diameter of the tube 15. An example of a typical existing retention member is the inflatable internal bolster disclosed in U.S. Pat. No. 6,077,243. The '243 patent discloses an inflatable internal bolster having an outside diameter of 1.00 inch. The inflated bolster is associated with a 20 French tube having an outside diameter of 0.264 inches. The outside diameter of the inflated balloon, therefore, is less than four times the outside diameter of the tube and has a ratio of 3.8:1. Similarly, the '243 patent describes an inflatable bolster having an outside diameter of 0.712 inches associated with a 14 French tube having an outside diameter of 0.180 inches at its outer surface—a ratio of 3.9:1.

FIG. 3 illustrates an internal bolster of a PEG tube well known in the art in position against an internal wall of a body component or cavity such as the stomach or colon. As illustrated in FIG. 3, the connective tissue attachments 50 that hold body components in their anatomical positions along with the structural integrity of the body component itself produce a resistance force 52 that can resist the stretching or re-positioning of the body component(s) against the inner surface of the body by a retention bolster 20. In addition, when accessing highly mobile body components such as the colon and its peristaltic movement, variations in forces and tension are exerted on the internal bolster and the medical access device. The tension or force 54 necessary to oppose the resistance force 52 and to secure the medical access device in an operable position is translated through the rigid structure of the existing retention bolster 21 and often through delicate structures of the body component being accessed. The amount of force 54 necessary to overcome the resistance force 52 opposing the re-positioning of the body component constitutes a substantial amount of localized pressure on the structural elements of the tissues of the body component applied through the smaller surface area of the existing bolster 21. (The size of the force arrows 52 and 54 illustrated in FIG. 3 generally quantifies the amount of force applied by the prior art bolster to these tissues.)

Problems are known to occur because of the localized pressure applied by the small surface area of retention members having an outside diameter less than four times the outside diameter of its tubular member. The substantial amount of pressure on the tissues of the body component can disrupt the function of the structural elements and can lead to complications arising from long-term implantation of a PEG or PEC tube. Some of these complications include pressure necrosis, colonic and gastric perforation, colocutaneous fistula, gastric outlet blockage and gastric bleeding, cellulitis, tube extrusion, and stomal leakage as reported in Lin H, Ibrahim H Z, Kheng J W, Fee W E, Terris D J. Percutaneous Endoscopic Gastrostomy: Strategies for Prevention and Management of Complications. Laryngoscope 2001; 111(10): 1847-1852.

Retention members having a fixed position which apply continual, direct pressure to the internal surface of a body component being accessed can cause pressure necrosis due to this focused pressure. Further, as percutaneous medical access device placement techniques have become increasingly common, medical access devices have been increasingly used for longer periods of time. As such, complications at the site of medical access device insertion have become increasingly common. Existing retention members which remain inflexibly clamped to maintain the medical access device in position during use, do not accommodate the unavoidable movements of the medical access device during this long period of time. As the medical access device is moved about over time, either accidentally or by attending medical personnel, additional pressure is often applied on the tissues at the site causing the retention member to dig into the tissues engaged by the retention member resulting in damage to the tissue in the form of tissue necrosis. Furthermore, in the process of positioning a rigid retention member against the internal wall of a body component and against the skin of a subject, thus drawing together the two retention members to secure the medical access device, compression and constriction of the structural elements within the wall of the body component can occur as described above.

Another disadvantage of the smaller diameter of an existing retention member is its limited ability to promote adhesion of the tissue layers traversed by the medical access device, such as adhesion between the external surface of the body component, and the internal surface of the body. As FIG. 4 illustrates, a small diameter bolster is only able to expose a small surface area of the external wall 41 of the body component and the internal wall 47 of the body to attempt to join the layers in a sealing relationship around the stoma 48 of the medical access device. As discussed above, the seal between layers is especially significant when accessing the non-sterile environment of a moving body component. If the layers of tissue traversed by the medical access device are not secured to one another, the layers are free to undergo lateral movement between the layers which compromises the positioning of the tube. Moreover, inadequate adhesion between the layers can cause leakage of the contents of the body component to occur beyond an attachment point 60 into, or out of, the tissues and spaces 62 surrounding the access site.

There is, therefore, a need to develop a retention member of a medical access device that maintains the role of securing the medical access device in place while improving the seal between the accessed region and the inside of the medical access device and reducing the likelihood of complications resulting from prolonged implantation of the medical access device.

### SUMMARY OF THE INVENTION

The present invention provides a device and a method for securing a medical access device in an operable position to selectively deliver substances to and/or from a desired location within a body. In particular, the present invention provides a retention member having an outer diameter that is at least four times the outer diameter of the tubular portion of the medical access device.

In one embodiment of the present invention, an internal retention member for securing or retaining a tubular member of a medical access device against an internal surface of a body has a maximum outer surface diameter that is at least four times greater than a fixed outer surface diameter of the tubular member of the medical access device. In one embodiment, the maximum outer diameter of the retention member is adapted to be reduced to accommodate insertion, removal and position adjustment of the medical access device.

One embodiment of the present invention includes a method of percutaneously accessing an internal component of a body. The method includes inserting a tubular member through a passage between an external surface of the body and an internal surface of the body. The method also includes deploying a distal retention member circumferentially attached to the tubular member to secure the tubular member in the passage. In this embodiment, an outer surface diameter of the distal retention member is at least four times greater than an outer surface diameter of the tubular member.

It is an object of the present invention to provide an improved internal retention member for a medical access device.

It is another object of the present invention to provide an internal retention member which minimizes the effects of the retention pressure on an internal surface of a body component being accessed.

It is another object of the present invention to provide an internal retention member which optimizes adhesion of the internal body component surfaces and the abdominal wall.

Additional features and advantages of the present invention are described in, and will be apparent from, the following Detailed Description of the Invention and the figures.

### BRIEF DESCRIPTION OF THE FIGURES

FIG. 1 illustrates a microscopic cross-sectional view of the layers of the lining of the colon.

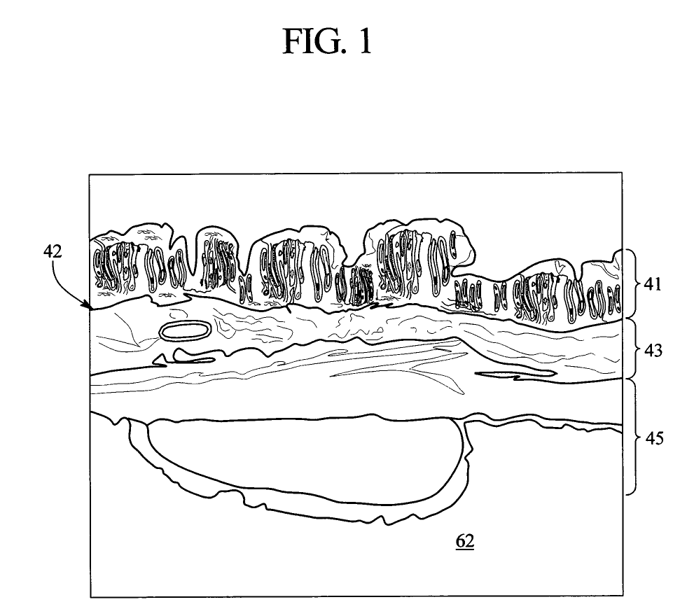

FIG. 2 illustrates a perspective view of a retention bolster known in the art.

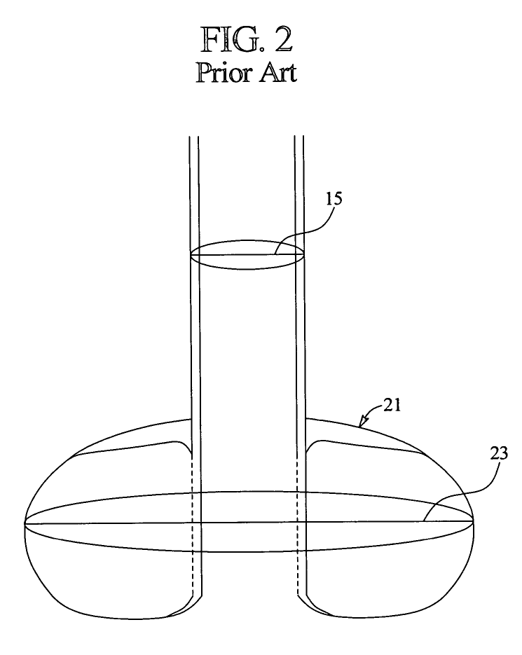

FIG. 3 illustrates a perspective view of the distal end of a retention bolster known in the art positioned within a subject.

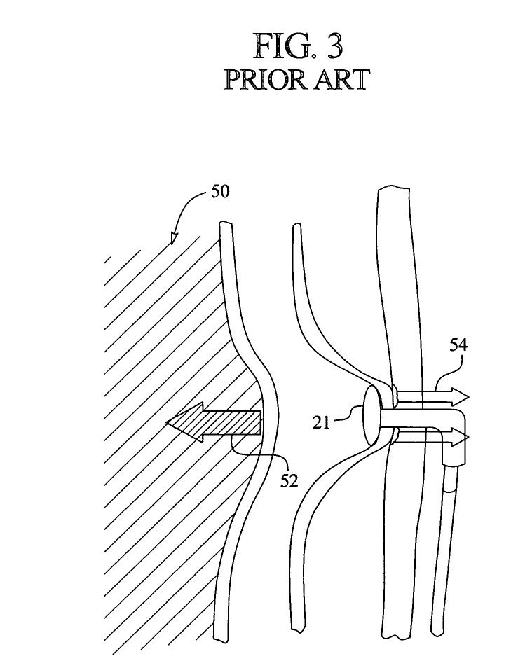

FIG. 4 illustrates a partial cross-sectional view of the interaction between layers effected by a retention bolster known in the art.

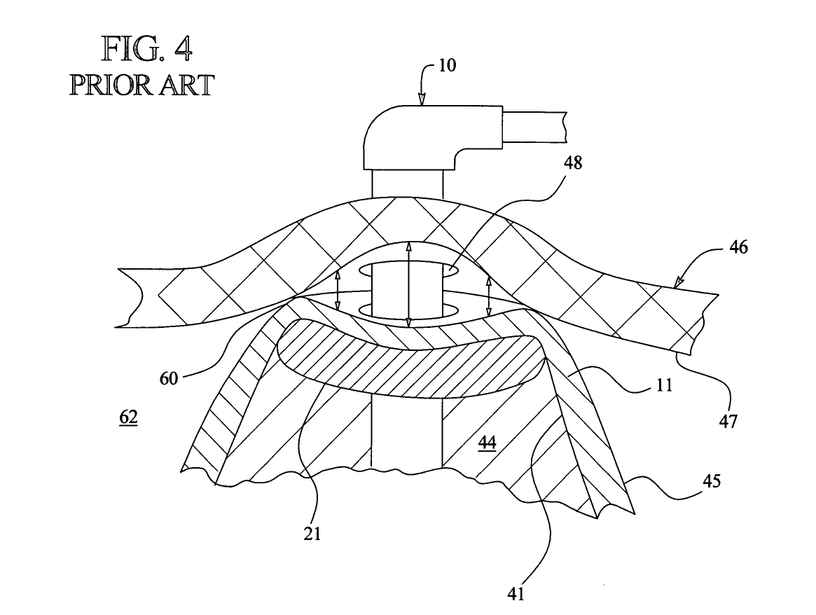

FIG. 5 illustrates a perspective view of the distal end of a retention member of one embodiment of the present invention.

FIG. 6 illustrates a partial cross-sectional view of the placement in a subject of a retention member positioned within one embodiment of the present invention.

FIG. 7 illustrates a partial cross-sectional view of the interaction between layers effected by a retention member of one embodiment of the present invention.

### DETAILED DESCRIPTION OF THE INVENTION

The present invention is related to a device and a method for securing a medical access device in an operable position to selectively deliver substances to and/or from a desired location in a body. In particular, the present invention includes an internal retention member for securing or retaining a medical access device against an internal surface of a body in a position to access an internal region of a body.

In one embodiment of the present invention, the medical access device includes a tubular member defining an inlet port and an outlet port and, at least one retention member associated with a distal end of the tubular member.

The medical access device 10 in one embodiment, is formed from a biocompatible polyurethane material. It should be appreciated that the medical access device 10 can be formed from other biocompatible materials including any biocompatible polymer or copolymer such as a silicone elastomer, silicone copolymer, thermoplastic rubber, or polyvinylchloride or mixtures thereof and can include components made of the same or different materials. The internal retention device, for example, can be formed from a dissolvable biocompatible material including any biocompatible dissolvable polymer materials or other materials known in the art, such as collagen, elastin, or chitin.

In addition to these materials the tubular member 12, in one embodiment, is reinforced by a stainless steel wire coil 42 embedded within the generally cylindrical wall of the tubular member 12. In one embodiment, the wire coil 42 extends from the level of the proximal end 16 of the tubular member 12 to a point approaching the distal end 18 of the tubular member 12. In one embodiment, the internal retention member 20 surrounds a portion of the tubular member 12 which is not supported by the wire coil 42.

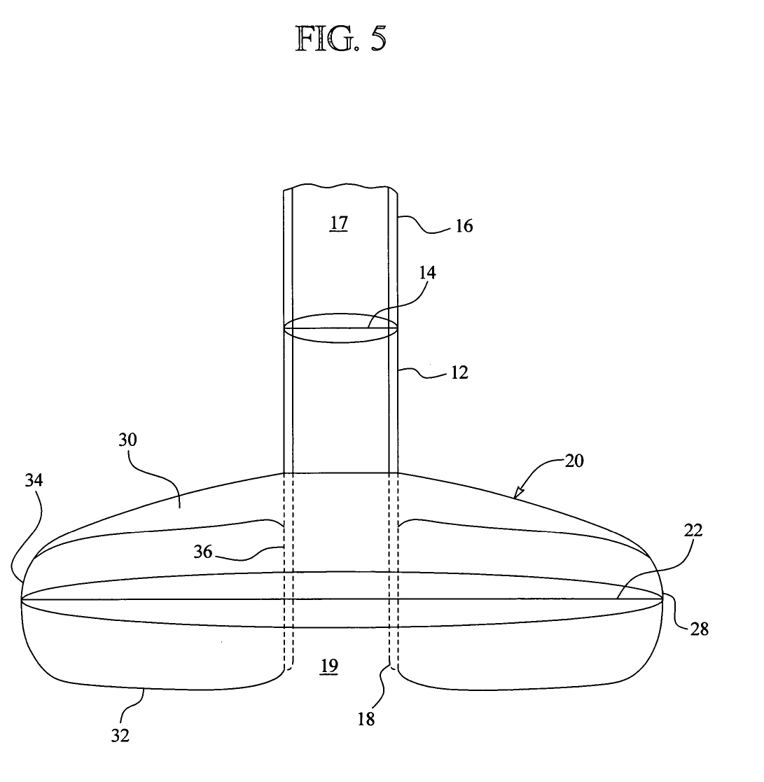

Referring now to FIG. 5, in one embodiment of the present invention, a medical access device 10 includes a tubular member 12 through which a substance is delivered to an internal body component being accessed. The tubular member 12 includes an outer tubular surface diameter 14, a proximal or external end 16 defining an inlet port 17 and a distal or internal end 18 defining an outlet port 19. It should be appreciated that any suitable outer tubular surface diameter sufficient to deliver the desired substance to the accessed body component can be used in the medical access device 10. It should also be appreciated that the proximal end 16 and the distal end 18 of the tubular member 12 can define an outlet 19 and an inlet port 17, respectively, through which a substance is delivered away from the internal body component.

In an embodiment in which a substance is delivered to an internal body component being accessed, the inlet port 17 at the proximal external end 16 of the tubular member 12 is connected in fluid communication with an administration assembly (not shown) containing the substance(s) to be delivered through the medical access device 10.

In the embodiment illustrated in FIG. 5, the present invention includes a retention member or bolster 20 having an outer retention member surface diameter 22 which is at least four times greater than the outer tubular surface diameter 14 of the tubular member 12 of the medical access device 10. It should be appreciated that, in one embodiment, reducing the outer tubular surface diameter allows the outer retention member surface diameter to be proportionally reduced. The retention member 20 is operably attached to the tubular member 12 of the medical access device 10 to secure or retain the medical access device 10 in a position to access a desired location in a body. In one embodiment of the present invention, an external retention member encircles the tubular member 12 at the proximal end 16 of the tubular member 12 to secure the tubular member 12 against the external surface of the body.

In one embodiment of the present invention, an internal retention member 20 encircles the tubular member 12 at the distal end 18 of the tubular member 12. In one embodiment, the internal retention member 20 is formed into a flattened spherical shape having substantially parallel proximal and distal surfaces 30 and 32, respectively, which are continuous with an outer sidewall 34 which curvedly joins, and is substantially perpendicular to, the proximal and distal surfaces 30 and 32. In addition, the proximal and distal surfaces 30 and 32 curvedly join, and are substantially perpendicular to an inner sidewall 36 which defines a diameter sized to allow the fitting of the bolster around the outer diameter of the tubular member 12. In one embodiment, this diameter is substantially equal to the outer tubular surface diameter 14. In one embodiment, the distal surface 32 is flush with the outlet port 19 preventing the tubular member 12 from protruding beyond the internal retention member 20 to present a flat or flush proximal surface 30 to the internal surface of the accessed body cavity such as the gastric mucosa of the stomach. It should be appreciated that the internal retention member 12 can include any suitable shape. For example, an internal retention member can include oval, circular or oblong shapes having concave, convex or substantially planar distal or proximal surfaces 30, 32. The shape of the retention member can also depend on the method of access and the anatomical constraints of the body component being accessed.

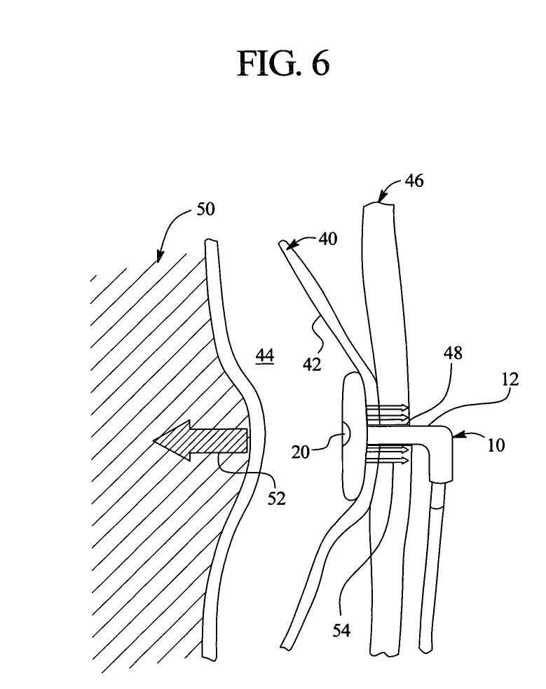

In the embodiment illustrated in FIG. 6, the retention member 20 is a preformed internal bolster operably associated with the distal or internal end 18 of the tubular member 12 of a percutaneous endoscopic colostomy (PEC) tube. The internal bolster 20 of the PEC tube 10 is adapted to engage an internal surface 42 surrounding a lumen 44 of the colon 40 being accessed by the PEC tube 10.

In FIG. 6, the PEC tube 10 is illustrated in a position in a subject in which the PEC tube 10 is secured in place by an internal bolster 20. The tubular portion 12 of the PEC tube 10 extends from outside the abdominal wall 46 of a subject through a stomal passage 48 to the lumen 44 of the colon 40 being accessed. The PEC tube 10 traverses the abdominal wall 46 and the wall of the colon 42 so that the bolster 20 and outlet port 19 are positioned within the colon lumen 44 of the subject. Although the invention is generally illustrated here in the context of a PEC tube, it may find equally advantageous application in other medical access devices such as a percutaneous endoscopic gastrostomy (PEG) tube, jejunostomy tube assemblies, suprapubic bladder catheter assemblies, etc.

The positioning of the PEC tube illustrated in FIG. 6, exposes the tube to a force 52 directed away from the bolster 20. The force 52 is generated by connective tissue attachments 50 opposite the access site along with the structural integrity of the body component itself. An equivalent force 54 opposes the connective tissue force 52 to retain the PEC tube 10 in position. The opposing force 54 is distributed over the surface area of the internal bolster 20 in contact with the wall 42 of the colon 40. The PEC tube 10 of the present invention has an increased surface area over which the opposing force 54 is distributed. In other words, the same opposing force 54 is distributed over a broader surface area of the bolster 20 of the present invention to reduce the pressure on the structures within the colon wall 42. Therefore, the pressure necessary to maintain the position of the PEC tube 10 in the subject is minimized by the improved bolster 20 of the present invention.

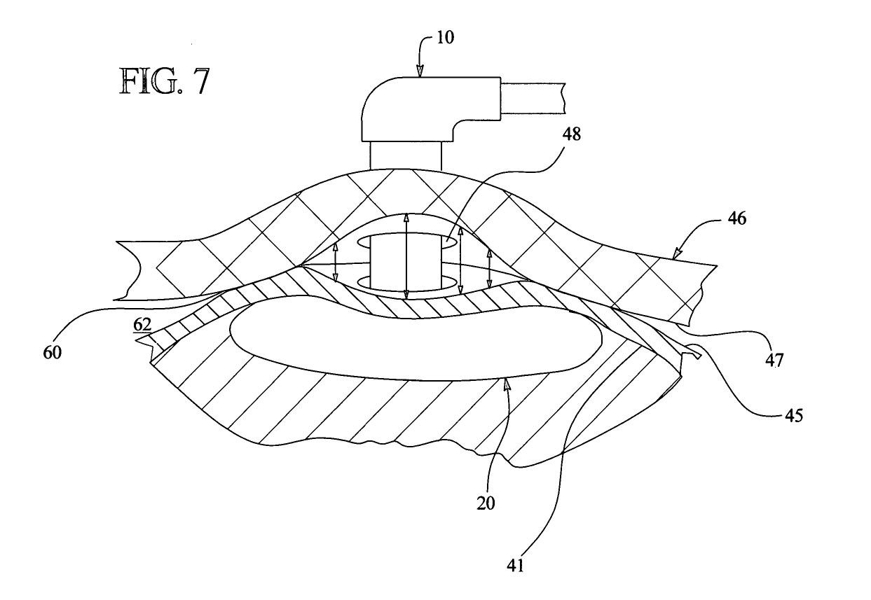

Referring now to FIG. 7, the expanded surface area of the internal bolster of one embodiment of the present invention, such as the PEC tube 10, also improves tissue adhesion between the internal surface of the abdominal wall 46 and the external surface of the colon wall 42. In order to minimize the likelihood of contamination from outside the body of the subject or leaking of the contents of the accessed body component into the surrounding tissues and spaces, the retention member of the present invention brings together the tissue layers, including the innermost and external layer through which the medical access device passes. As discussed above, bringing together the layers traversed by the tubular member 12 of the medical access device 10 minimizes lateral movement between the layers that compromise the positioning of the tube. The promotion of tissue adhesion between the layers traversed by the PEC tube 10 of the present invention also contributes to the formation of a better seal between the space being accessed and the inside of the tube. The increased surface area of the internal bolster 20 enables a larger surface area of the external surface of the colon wall 42 to be exposed to the internal surface of the abdominal wall 46. Contact between the two surfaces 42 and 46 activates factors that stimulate the formation of connective tissue or adhesions to eventually form an anatomic seal between the adjoined layers 42 and 46. This biological seal minimizes the likelihood of the contents of the colon to leak into the surrounding peritoneal space and other tissues where infection can occur.

In one embodiment, the internal retention member is a substantially solid or non-inflatable bolster substantially resistant to deformation. Alternatively, the internal retention member 20 is an inflatable bolster which forms a pre-formed shape upon active or passive inflation with a liquid or gas, such as air, at ambient pressure. The rigidity of the internal retention device is sufficient to prevent the medical access device 10 from being inadvertently pulled out of an orifice, passage or stoma without substantial effort.

In one embodiment, the internal retention member of the present invention is designed to undergo deformation to allow insertion and/or retraction of the medical access device through an orifice, passage or stoma. In one embodiment, when the internal retention member is in its pre-formed retention configuration, force applied to the flat retention surface of the internal retention member tends to deform the internal retention member. In one embodiment, deformation of the retention member allows removal of the medical access device from the accessed body component by firm outward traction on the medical access device. In one embodiment, the retention member is adapted to be passed naturally through an orifice of the body. It should be appreciated that insertion and retraction of internal retention members having increased outer diameter can also be accommodated by collapse and/or deflation of a pre-formed bolster through at least one deflation lumen 38 incorporated along the length of the tubular member 12 or by other suitable means known in the art. Examples of the adaptation of an internal retention member to be inserted, advanced and/or retracted while maintaining an ability to be expanded, are described in U.S. Pat. Nos. 4,981,471 and 5,556,385 incorporated herein by reference.

### EXAMPLE

In one embodiment of the present invention, an internal retention bolster having an outside diameter at its outer surface of approximately 0.900 inches is associated with a PEC tube having an outside diameter of approximately 0.160 inches (12 French). Therefore, the outside diameter of the bolster is 5.625 times the outside diameter of the PEC tube.

It should be appreciated that the proportions of the enlarged internal retention bolster of the present invention can be adapted to be associated with any size tubular member of any type of access device. In one embodiment, the diameter of the tubular member is based on the flow requirements and flow characteristics of the infusion material to be passed through the tubular member. For example, a tubular member delivering a viscous solution, such as an enteral nutrition formula, may require a greater diameter than a tubular member delivering a less viscous solution, such as an irrigant, if the same rate of flow under the same amount of pressure is desired. Accordingly, the diameter of the PEC tube is not necessarily dependent on the size of the subject.

It should be understood that various other changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A medical access device for accessing a portion of a body comprising:
a tubular member having an outer tubular diameter; and
a retention member circumferentially attached to the tubular member having an outer retention member diameter, wherein the outer retention member diameter is at least four times greater than the outer tubular diameter.

2. The medical access device of claim 1, wherein the retention member is substantially associated with a distal portion of the tubular member, said distal portion being positioned within the body.

3. The medical access device of claim 1, wherein the retention member includes a substantially flattened spherical shape.

4. The medical access device of claim 1, wherein the ratio of the outer retention member diameter to the outer tubular diameter is at least 5:1.

5. The medical access device of claim 1, which includes a percutaneous endoscopic colostomy tube.

6. The medical access device of claim 1, wherein the retention member is adapted to be inserted through at least one tissue layer in a non-deployed configuration such that the outer retention member diameter is decreased.

7. The medical access device of claim 1, wherein the retention member is expandable by a gas.

8. The medical access device of claim 1, wherein the retention member is adapted to be removed by traction through at least one tissue layer in a non-deployed configuration such that the outer retention member diameter is decreased.

9. The medical access device of claim 1, which includes an external retention member.

10. A method of percutaneously accessing a body component comprising:
(a) providing a medical access device including a tubular member having an outer tubular diameter and a retention member having an outer retention member diameter;
(b) inserting the tubular member through a passage between an external surface of a body and an internal surface of the body; and
(c) securing the medical access device against the internal surface of the body by deploying a distal retention member circumferentially attached to the tubular member, wherein the outer retention member diameter is at least four times greater than the outer tubular diameter.

11. The method of claim 10, which includes substantially associating the retention member with a distal portion of the tubular member, said distal portion being positioned within the body.

12. The method of claim 10, wherein the retention member includes a substantially flattened spherical shape.

13. The method of claim 10, which includes securing the medical access device against the external surface of the body by deploying a retention member circumferentially attached to the tubular member.

14. The method of claim 13, wherein securing the medical access device against the external surface of the body includes a retention member having an outer retention member diameter at least four times greater than the outer tubular diameter.

15. The method of claim 10, wherein the ratio of the outer retention member diameter to the outer tubular diameter is at least 5:1.

16. The method of claim 10, which includes a percutaneous endoscopic colostomy tube.

17. The method of claim 10, which includes adapting the retention member to be inserted through the passage in a non-deployed configuration such that the outer retention member diameter is decreased.

18. The method of claim 10, wherein deploying the distal retention member includes expanding said distal retention member with a gas.

19. The method of claim 10, which includes adapting the retention member to be removable through the passage by traction in a non-deployed configuration such that the outer retention member diameter is decreased.
